(19) United States
(12) Patent Application Publication
(10) Pub. No.: US 2003/0004352 A1
(43) Pub. Date: Jan. 2, 2003

(54) Novel process

(76) Inventors: Christopher S. Brook, Havertown, PA (US); Alan D. Curzons, Worthing (GB); Carolyn W. Grady, Philadelphia, PA (US); Anthony J. O'Connor, Worthing (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/149,527

(22) PCT Filed: Dec. 22, 2000
(86) PCT No.: PCT/US00/35109

(30) Foreign Application Priority Data
Dec. 23, 1999 (GB) 9930577.3

(51) Int. Cl. C07D 211/22

(52) U.S. Cl. 546/240

(57) ABSTRACT

A process for the manufacture of the (−) trans piperidine carbinol (1) by a process comprising contacting a racemic mixture of the piperidine carbinol in solution with (−)-ditoluoyltartaric acid, crystallising the (−) -ditoluoyltartaric acid salt of the piperidine carbinol, isolating the crystalline salt and neutralising the crystalline salt to regenerate the (−) trans isomer of the piperidine carbinol and the (−)-ditoluoyltartaric acid, which is characterised by one or more of the following steps: (1) combining solutions of the racemic piperidine carbinol and (−)-ditoluoyltartaric acid in acetone so that the combined solution contains 2-3 % wt/wt of water, (2) consolidating the chiral salt crystallisation at from 30 to 40° C., (3) cooling the crystallisation mixture to from 3 to 7° C. before isolating the chiral salt, (4) regenerating the (−) trans piperidine carbinol at a pH of from 10.5 to 11.5, (5) forming a concentrated solution of the (−) trans piperidine carbinol in toluene, contacting the solution with heptane at 60-65 ° C., and cooling stepwise to crystallise the (−) trans piperidine carbinol. Alternatively, a solution of the racemic piperidine carbinol in toluene, suitably from a previous stage in the manufacture of paroxetine, is combined with a solution of (−)- ditoluoyltartaic acid in acetone. The resultant (−) trans piperidine carbinol of structure (1) may be coupled with sesamol, then deprotected, to give paroxetine (2), with optional formation of a pharmaceutically acceptable salt of paroxetine.

This invention relates to processes for the manufacture of intermediates for paroxetine and pharmaceutically acceptable salts thereof which are suitable for large scale commercial operation.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxyphenoxymethyl)-piperidine. This compound is used in therapy as the hydrochloride salt to treat inter alia depression, obsessive compulsive disorder (OCD) and panic.

Various processes have been described for the preparation of paroxetine, for example in U.S. Pat. No. 4,007,196, EP 0219,934, EP 0223,334, EP 223,403, EP 0300,617 and Acta Chemica Scandinavica (1996) volume 50 page 164. A particularly useful starting material employed in processes described therein is (−) trans 4-(4′-fluorophenyl)-3-hydroxymethyl-1-methylpiperidine, which is the (−) form of the trans-piperidine carbinol of structure (1)

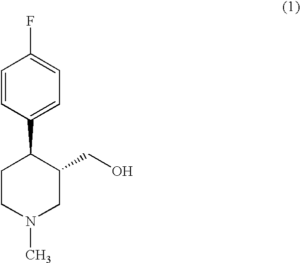

In the process described in EP 0223334, a racemic trans-piperidine carbinol of structure (1) is resolved by conversion to a salt with a chiral acid. In Example 5 of EP 0223334 (+)-2′-nitrotartranilic acid is used, and in Example 8 the resolving agent is (−)-di-p-toluoyltartaric acid. The (−) trans carbinol is subsequently liberated from the chiral acid salt and may then be coupled with sesamol, then deprotected, to give paroxetine (2).

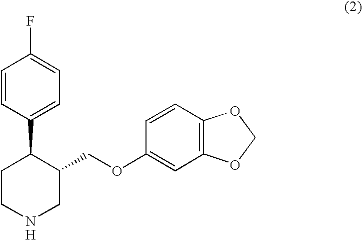

The chiral acid resolving agent specified in Example 5 of EP 0223334, (+)-2′-nitrotartranilic acid, is not commercially available, and the synthesis of this acid requires a number of steps. The process of Example 5 is therefore not suited to large scale manufacture.

The (−)-di-p-toluoyltartaric acid employed in Example 8 of EP 0223334 is commercially available, but we have found that the process described in Example 8 is extremely difficult to control on a large scale, giving erratic yields and frequent failure to meet specification. A major problem which has been encountered is co-crystallisation of the (−)-ditoluoyltartaric acid salt of the unwanted (+) trans carbinol isomer.

We have also found that use of Example 8 conditions for the regeneration of (−) trans carbinol from the (−)-ditoluoyltartaric acid salt on a large scale leads to unacceptable levels of impurities. Among these are ditoluoyltartaric acid, monotoluoyltartaric acid, toluic acid and tartaric acid. We have also found that the isolation conditions specified in Example 8 frequently lead to degradation and/or an oily product.

This invention provides improved processes for the preparation of the (−) trans piperidine carbinol of structure (1) in which the levels of these and other impurities are controlled within acceptable limits, enabling the preparation of paroxetine from a compound of structure (1) to be carried out on a manufacturing scale.

During the preparation of pharmaceutically active compounds on a manufacturing scale, small variations in procedures and reactants may have a significant effect on the commercial viability of the process. The problems that need to be addressed in commercial production, and the solutions to the problems, are not predictable during the scaling up of a process that is successful on a laboratory scale. Commercial scale operation typically involves use of reactants in kilogram quantities, usually in tens of kilograms, for example at least 30 kg, more usually in hundreds of kilograms, for example 300-1000 kg. Similarly the reactor vessels used are typically at least 100 litres in capacity, more usually from 1-10 kilolitres.

Thus the present invention has been developed with the aim of improving the preparation of the (−) form of the trans-piperidine carbinol of structure (1) by a resolution process with a chiral acid, more particularly by contacting a racemic trans piperidine carbinol of structure (1) in solution with (−)-ditoluoyltartaric acid, crystallising the (−)-ditoluoyltartaric acid salt of the piperidine carbinol, isolating the crystalline salt and neutralising the crystalline salt to release the (−) trans isomer of the piperidine carbinol and regenerate the (−)-ditoluoyltartaric acid. The improvements to this process provided by the present invention are of significant value in a commercial process for the large scale manufacture of the (−) trans carbinol of formula (1).

The improved process of the present invention is characterised by one or more of the following steps:

(1) combining acetone solutions of the racemic piperidine carbinol and (−)-ditoluoyltartaric acid, typically in aqueous acetone and anhydrous acetone respectively, so that the combined solution contains 2-3 % wt/wt of water,

(2) combining a toluene solution of the racemic piperidine carbinol, or optionally a solution thereof in toluene and an auxiliary solvent, typically acetone, and a solution of (−)-ditoluoyltartaric acid in a compatible solvent, typically acetone,

(3) initiating the chiral salt crystallisation at from 30 to 40° C.,

(4) cooling the crystallisation mixture to from 3 to 7° C. before isolating the chiral salt,

(5) regenerating and extracting the (−) trans piperidine carbinol at a pH of from 10.5 to 11.5,

(6) forming a concentrated solution of the (−) trans piperidine carbinol in toluene, contacting the solution with heptane at 60-65° C., and cooling stepwise to crystallise the (−) trans piperidine carbinol.

The various aspects of the improved process of the present invention are discussed below with reference to the key technical areas:

i) The Presence of Water in Acetone Solutions

Example 8 of EP 0223 334 uses acetone as solvent and specifies anhydrous conditions. We have surprisingly found that the presence of some water in the acetone is beneficial, though the amount of water present must be controlled within narrow limits.

We have found that formation of the (−)-ditoluoyltartaric acid salt of the (−) trans carbinol in acetone containing up to 2% wt/wt of water avoids precipitation of the undesired salt of the (+) trans carbinol. Additionally we have found that the presence of water is necessary to achieve rapid and complete dissolution of the racemic trans carbinol. We have found that both requirements can be met by the convenient procedure of making a single addition of 4-6% wt/wt water to the acetone used to form the solution of the racemic carbinol.

Thus in the improved process the racemic trans piperidine carbinol of structure (1) is preferably dissolved in acetone containing 4 to 6% wt/wt of water and the (−)-ditoluoyltartaric acid is preferably dissolved in the same number of litres of substantially anhydrous acetone, so that the crystallisation mixture contains from 2 to 3% wt/wt of water.

For example this may be achieved by dissolving trans 4-(4′-fluorophenyl)-3-hydroxymethyl-1-methylpiperidine in preferably 8 to 10 litres of acetone per kg, the acetone preferably containing between 4 and 6 % wt/wt of water, preferably at 30-35° C., and separately dissolving preferably 1.6 to 1.7 molar equivalents (relative to the carbinol) of (−)ditoluoyltartaric acid in preferably 2 to 4 litres of anhydrous acetone per kg preferably at 30-35° C.

ii) Initial Crystallisation Temperature

Example 8 of EP 0223 334 specifies a temperature of 15-25° C. for the initial crystallisation of the chiral acid salt. We have surprisingly found that the use of elevated temperatures results in a much more robust process on a large scale, and gives more reliable results.

In the improved process the initial chiral salt crystallisation is preferably carried out at between 30 and 40° C., more preferably between 35 and 40° C. The optimum initial crystallisation temperature is conveniently achieved by mixing a warm solution of the carbinol with a warm solution of the chiral acid, and allowing the exothermic reaction to bring the temperature into the desired range.

This may be achieved by bringing together the trans 4-(4′-fluorophenyl)-3-hydroxymethyl-1-methylpiperidine and (−) ditoluoyltartaric acid solutions with rapid stirring, suitably at 120 rpm, suitably as quickly as possible although a mixing time of 0.5-2.5 hours may be convenient, preferably at a temperature between 30 and 40° C., more preferably between 35 and 40° C., and holding the crystallisation mixture at preferably between 35 and 40° C. for preferably 30 minutes to consolidate the crystallisation. Preferably the crystallisation is completed by cooling the crystallisation mixture to between 3 and 7° C., for preferably 0.5 to 1.5 hours, more preferably for 1 hour, isolating the ditoluoyltartaric acid salt preferably at between 3 and 7° C., washing with acetone, and optionally drying the ditoluoyltartaric acid salt,

iii) pH Used to Regenerate Carbinol from the Salt

Example 8 of EP 0223 334 does not specify the amount of sodium hydroxide to be added, or the pH which is required. We have found that the pH at this point is critical on the large scale, as the extended reaction times arising from large scale operation cause hydrolysis of the ditoluoyltartaric acid, generating monotoluoyltartaric acid, toluic acid and tartaric acid. These are troublesome impurities, which are difficult to remove. Furthermore this hydrolysis reaction destroys (−)-ditoluoyltartaric acid, which could otherwise be recovered and re-used, adding significantly to the cost of manufacture.

In the improved process the pH at this point is preferably in the range 10.5 to 11.5, more preferably in the range 10.8 to 11.2

For example this may be achieved by dissolving the ditoluoyltartaric acid salt in water by the addition of an inorganic base, suitably aqueous sodium hydroxide, and extraction of the (−) trans carbinol into an organic solvent, which may be dichloromethane as in Example 8 of EP 0223 334, or more conveniently toluene, preferably at a temperature in the range 15 to 25° C., at a pH preferably in the range 10.5 to 11.5, more preferably in the range 10.8 to 11.2,

iv) Crystallisation and Isolation Conditions

In the process described in Example 8 of EP 0223 334, a toluene solution is evaporated to give an oil which is triturated with heptane to give a crystalline solid. We have found that attempts to use this procedure on a large scale result in an impure product of poor crystalline form, which is difficult to isolate and dry.

In the improved process, a concentrated toluene solution is contacted with heptane at elevated temperature, preferably at 60-65° C., then cooled stepwise, preferably holding the temperature firstly at 40° C. to initiate crystallisation, with optional seeding, then at 20° C., and finally at 0° C. This gives a good crystalline form of the product which has low levels of impurities and can be readily isolated and dried.

Typically the improvement comprises concentrating the solvent (e.g. dichloromethane) extract by evaporation of solvent under reduced pressure, preferably at less than 50° C., and replacing the solvent with toluene. Alternatively toluene itself may be used as the extraction solvent. This is followed by concentrating the toluene solution, preferably at less than 65° C., and mixing with heptane, preferably at 60-64° C., then cooling slowly, preferably to 40° C. over 1 hour to initiate crystallisation, optionally seeding, further cooling slowly, preferably to 20° C. over 1 hour, and finally cooling, preferably to 0° C. The crystalline (−) trans 4-(4′-fluorophenyl)-3-hydroxymethyl-1-methylpiperidine is preferably collected at 0° C. , washed with heptane, which may be chilled if required, for example to 0-5° C., and dried at preferably less than 35° C. until the loss in drying is preferably less than 0.5%.

Optionally all or part of the (−)ditoluoyltartaric acid employed in step (1) or (2) above may be recovered from previous reaction liquors.

In a preferred embodiment, the stirring rate is significantly reduced during cooling down at step (4), suitably to 50 rpm, suitably when the temperature has fallen to 20° C.

In a particularly useful process variation, the ditoluoyltartaric acid salt isolated at step (4) is carried forward to step (5) as a wet cake containing acetone.

Optionally, the extraction solvent used in step (5) is mixed with the aqueous phase prior to the addition of the ditoluoyltartaric acid salt.

In a further useful process variation, the solvent extraction at step (5) is carried out using a centrifugal extractor.

Optionally a toluene solution of the desired (—) trans carbinol may be carried forward to the next step in the synthesis of paroxetine, by omitting the procedures of addition of heptane and crystallisation described in step (6). Such a process variation is especially useful for the large scale production of paroxetine.

Therefore in essence the present invention provides a process for the large scale manufacture of the (−) trans carbinol of formula (1) by a resolution process with a chiral acid which comprises the following steps

a) forming respective solutions in acetone of trans 4-(4′-fluorophenyl)-3-hydroxymethyl-1-methylpiperidine and (−)ditoluoyltartaric acid, or of trans 4-(4′-fluorophenyl)-3-hydroxymethyl-1-methylpiperidine in toluene and (−)ditoluoyltartaric acid in acetone,

b) bringing together the trans 4-(4′-fluorophenyl)-3-hydroxymethyl-1-methylpiperidine and (−)ditoluoyltartaric acid solutions with stirring,

c) holding the mixture to consolidate the crystallisation,

d) cooling the crystallisation mixture, isolating the ditoluoyltartaric acid salt and washing with acetone, and optionally drying the ditoluoyltartaric acid salt,

e) dissolving the ditoluoyltartaric acid salt in water by the addition of an inorganic base, and extracting the (−) trans carbinol into an organic solvent,

f) concentrating the solvent extract by evaporation of solvent under reduced pressure, and replacing the solvent with toluene,

g) concentrating the toluene solution, and mixing with heptane,

h) cooling slowly to initiate crystallisation,

i) collecting the crystalline (−) trans 4-(4′-fluorophenyl)-3-hydroxymethyl-1-methylpiperidine, washing with heptane, and drying.

More specifically in one aspect the process of the present invention is characterised by one or more of the following steps:

a)i) dissolving trans 4-(4′-fluorophenyl)-3-hydroxymethyl-1-methylpiperidine in acetone with added water, which preferably contains between 4 and 6 % wt/wt of water, preferably at 30-35° C, and

ii) separately dissolving preferably 1.6 to 1.7 molar equivalents of (−)ditoluoyltartaric acid in preferably substantially the same volume of acetone as used in step a)i), preferably at 30-35° C.,

b) bringing together the trans 4-(4−-fluorophenyl)-3-hydroxymethyl- 1-methylpiperidine and (−)ditoluoyltartaric acid solutions with rapid stirring, suitably at 120 rpm, preferably at a temperature between 30 and 40° C., more preferably between 35 and 40° C.,

c) holding the crystallisation mixture at a temperature between 30 and 40° C. preferably between 35 and 40° C. for preferably 30 minutes to consolidate the crystallisation,

d) cooling the crystallisation mixture preferably to between 3 and 7° C., for preferably 0.5 to 1.5 hours, more preferably for I hour, isolating the ditoluoyltartaric acid salt preferably at between 3 and 7° C., washing with acetone, and optionally drying the ditoluoyltartaric acid salt,

e) dissolving the ditoluoyltartaric acid salt in water by the addition of an inorganic base, suitably aqueous sodium hydroxide, and extraction of the (−) trans carbinol into an organic solvent, suitably dichloromethane, preferably at a temperature in the range 15 to 25° C., at a pH preferably in the range 10.5 to 11.5, more preferably in the range 10.8 to 11.2,

f) concentrating the solvent extract by evaporation of solvent under reduced pressure, preferably at less than 50° C., and replacing the solvent with toluene,

g) concentrating the toluene solution, preferably at less than 65° C., and mixing with heptane, preferably at 60-64° C.,

h) cooling slowly, preferably to 40° C. over 1 hour to initiate crystallisation, optionally seeding, further cooling slowly, preferably to 20° C over 1 hour, and finally cooling, preferably to 0° C.,

i) collecting the crystalline (−) trans 4(4′-fluorophenyl)-3-hydroxymethyl- 1-methylpiperidine preferably at 0° C., washing with heptane, and drying at preferably less than 35° C. until the loss in drying is preferably less than 0.5%.

The racemic trans carbinol of structure (1) used as one of the starting materials of the process of this invention may be prepared as described in Example 7 of EP 0223334.

However in an advantageous variant of the process of this invention, we have surprisingly found that all or part of the acetone used to dissolve the racemic trans carbinol in the published processes may be replaced by toluene. This finding is especially advantageous, firstly because toluene is an especially suitable solvent for use in commercial manufacturing processes and it is useful to reduce the amount of acetone used; secondly because the racemic trans carbinol starting material may be then be used as a toluene solution derived from a previous stage in the commercial manufacture of paroxetine, avoiding the need to isolate the racemic carbinol. Thus, as in step (2) and step (a) above, a solution of the racemic carbinol in toluene or a mixture of acetone and toluene may be combined with a solution of ditoluoyltartaric acid in acetone.

We have found that satisfactory formation and recovery of the desired (−) trans carbinol salt can be achieved by substituting toluene for acetone in conventional solutions of the racemic carbinol by adding from 1 to 3 volumes of toluene (with respect to the carbinol) to an acetone solution of the carbinol, and by using a wholly toluene solution of the carbinol for combining with an acetone solution of the chiral acid at a volume ratio of toluene:acetone of 1:1.

A toluene solution of the racemic trans carbinol may be prepared for example by treatment of an imide ester with lithium aluminium hydride (as described in Example 7 of EP 0223334) by omitting the final treatment with petroleum ether, and the isolation, washing and drying of the solid product.

In the same way that toluene may be used as a partial replacement for acetone in the conventional processes, so an auxiliary solvent, such as acetone, may be added to a toluene solution of the carbinol for compatibility with an acetone solution of the chiral acid. Also water may be added, for the reasons discussed above, i.e. to reduce the risk of precipitation of the undesired salt of the (+) trans carbinol.

Using this variant of the process for combing solutions of the racemic carbinol and the chiral acid, the ditoluoyltartaric acid salt of the (−) trans carbinol may then be isolated and processed to liberate the (−) trans carbinol in accordance with the remaining steps of the process sequences disclosed by this invention.

The (−) trans carbinol compound of structure (1) obtained by the processes of this invention may be converted to the active compound paroxetine using conventional procedures disclosed in U.S. Pat. No. 3912743, U.S. Pat. No. 4007196 or EP-A-0223334 whereby the (−) trans carbinol is coupled with sesamol, then deprotected, to give paroxetine (2).

The present invention includes within its scope the compound paroxetine, and paroxetine salts such as paroxetine hydrochloride, especially as an anhydrate or the hemihydrate, when obtained via any aspect of this invention.

The resultant paroxetine is preferably obtained as or converted to a pharmaceutically acceptable derivative such as a salt, more especially the methanesulphonate salt or the hydrochloride salt and most preferably the hemihydrate of that salt, as described in EP-A-0223403. Paroxetine free base may be converted to paroxetine methanesulphonate by treatment with methansulphonic acid or a labile derivative thereof, for example a soluble salt such as ammonium methanesulphonate. Paroxetine hydrochloride may be prepared by treatment of paroxetine free base with a source of hydrogen chloride, for example gaseous hydrogen chloride, or a solution thereof, or aqueous hydrochloric acid.

Paroxetine and its salts obtained using this invention may be formulated for therapy in the dosage forms described in EP-A-0223403 or WO96/24595, either as solid formulations or as solutions for oral or parenteral use.

Therapeutic uses of paroxetine, especially paroxetine hydrochloride, obtained using this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Accordingly, the present invention also provides:

a pharmaceutical composition for treatment or prophylaxis of the Disorders comprising paroxetine or paroxetine salt obtained using the process of this invention and a pharmaceutically acceptable carrier;

the use of paroxetine or paroxetine salt obtained using the process of this invention to manufacture a medicament for the treatment or prophylaxis of the Disorders; and

a method of treating the Disorders which comprises administering an effective or prophylactic amount of paroxetine or paroxetine salt obtained using the process of this invention to a person suffering from one or more of the Disorders.

Pharmaceutical compositions using active compounds prepared in accordance with this invention are usually adapted for oral administration, but formulations for dissolution for parental administration are also within the scope of this invention.

The composition is usually presented as a unit dose composition containing from 1 to 200 mg of active ingredient calculated on a free base basis, more usually from 5 to 100 mg, for example 10 to 50 mg such as 10, 12.5, 15, 20, 25, 30 or 40 mg by a human patient. Most preferably unit doses contain 20 mg of active ingredient calculated on a free base basis. Such a composition is normally taken from 1 to 6 times daily, for example 2, 3 or 4 times daily so that the total amount of active agent administered is within the range 5 to 400 mg of active ingredient calculated on a free base basis. Most preferably the unit dose is taken once a day.

Preferred unit dosage forms include tablets or capsules, including formulations adapted for controlled or delayed release.

The compositions of this invention may be formulated by conventional methods of admixture such as blending, filling and compressing. Suitable carriers for use in this invention include a diluent, a binder, a disintegrant, a colouring agent, a flavouring agent and/or preservative. These agents may be utilised in conventional manner, for example in a manner similar to that already used for marketed anti-depressant agents.

This invention is illustrated by the following Example.

### EXAMPLE 1

### a) Manufacture of (—)-ditoluoyltartaric Acid Salt of (−) Trans 4-(4′-fluorophenyl)-3-hydroxymethyl-1-methylpiperidine

A 1600 L glass-lined reactor is purged with nitrogen and charged with acetone (830 kg) and water (46 L). Trans 4-(4′-fluorophenyl)-3-hydroxymethyl- 1-methylpiperidine (110.4 kg at 100%) is then added, and the contents of the vessel are heated to 30 - 33° C. with good agitation until a clear solution is obtained (1 hour).

A 2250 L glass lined reactor is purged with nitrogen and charged with acetone (830 kg), followed by (−)-ditoluoyltartaric acid (320.0 kg at 100%). The mixture is agitated at 120 rpm at 30-33° C. until a clear solution is obtained, then the trans 4-(4′-fluorophenyl)-3-hydroxymethyl-1-methylpiperidine solution is added, holding the temperature between 30 and 40° C. The addition time should be between 30 minutes and 2.5 hours. The vessel containing the (−) carbinol solution is rinsed with acetone (16 kgs) and the rinse is transferred to the main reactor.

The temperature of the 2250 L reactor is held between 35 and 40° C. for 30 minutes, then lowered to 20° C. At this point the agitator speed is reduced to 50 rpm, then mixture is further cooled to 5° C. and held at this temperature for 30 minutes.

The (−)-ditoluoyltartaric acid salt is collected using a stainless steel nitrogen purged centrifuge, and washed with 1.1 kg acetone per kg of wet product. The crystallisation vessel is rinsed with acetone (40 kg) which is also used to wash the product.

### b) Isolation of (−) Trans 4-(4′-fluorophenyl)-3-hydroxymethyl-1-methylpiperidine

A 6300 L glass-lined reactor is charged with dichloromethane (2060 kg) and water (1555 L), followed by the (−)-ditoluoyltartaric acid salt wet-cake (4 batches as manufactured above). Further dichloromethane (2060 kg) and water (1555 L) are added, followed by 25% wt/wt aqueous sodium hydroxide (220 L), maintaining the temperature in the range 15 to 25° C.

The mixture is agitated at 150 rpm for 20 minutes, then the pH is measured and adjusted if necessary to within the range 10.8 to 11.2 using 1 kg aliquots of 25% wt/wt sodium hydroxide. The mixture is agitated for 30 minutes at 15-25° C., and the pH again measured and adjusted if necessary to 10.8 to 11.2 by addition of sodium hydroxide.

The phases are separated and the lower dichloromethane layer retained. The aqueous phase is further extracted with dichloromethane (2000 kg), again checking and adjusting the pH as before.

The combined dichloromethane layers are agitated with water (2000 L) for 15 minutes in a 6300 L vessel, again holding the pH at 10.8 to 11.2, if necessary by the addition of sodium hydroxide

The dichloromethane layer is separated and agitated with water (2000 L) for 30 minutes. The aqueous phase is discarded and the dichloromethane phase is transferred to a thoroughly clean 6300 L vessel, washing in with further dichloromethane (265 kg). The dichloromethane phase is cooled to 10° C., and distilled under reduced pressure to a volume of 535 L at a temperature not exceeding 40° C.

When the distillation is complete, toluene (1223 kg) is added and the mixture distilled under reduced pressure to a volume of 430 L at a temperature not exceeding 65° C., the final temperature being achieved by ramping from 25 to 65° C. over 1 hour.

n-Heptane (1233 kg) is charged to a 2200 L stainless steel holding vessel and warmed to 60 to 64° C., then added to the concentrated toluene solution at 60-64° C., with the agitator set at 90 rpm. The mixture is cooled to 40° C. over 1 hour, when crystallisation either commences spontaneously, or is initiated by seeding. The slurry of crystals is then cooled to 20° C. over 1 hour, and further cooled to 0° C. over 2 hours.

The product is collected in a 1 M² filter drier, washed with chilled n-heptane (212 kg) and blown down with nitrogen until the loss on drying is less than 15%. Heating is then applied and the product is dried at a maximum temperature of 35° C. until the loss on drying is less than 0.5% (4 hours).

The cake is cooled to less than 32° C before discharge into kegs.

The yield is about 176 kgs.

### EXAMPLE 2

### (31 ) Trans 4-(4′-fluorophenyl)-3-hydroxymethyl-1-methylpiperidine

i) Acetone (476 ml) and water (21 ml) are added to a mixture of racemic trans 4-(4′-fluorophenyl)-3-hydroxymethyl-1-methylpiperidine (50 g) in toluene (50 ml) and warmed to 33° C. to give a clear solution. This is added to a solution of (−)-ditoluoyltartaric acid (146 g) in acetone (476 ml), previously warmed to 33° C., and the mixture stirred at 35-40° C. for 30 minutes. The slurry of crystals is then cooled to 5° C., and stirred at this temperature for 1 hour. The salt is collected and washed with cold acetone ( 2×30 ml).

ii) The wet cake from part i) is stirred in water (200 ml) and the pH adjusted to 8.5-9.0 using 25% aqueous sodium hydroxide, then to 10.8-11.2 using 5% sodium hydroxide. Dichloromethane (200 ml) is added, and if necessary the pH is re-adjusted to between 10.8 and 11.2 using 5% sodium hydroxide. The phases are separated, and the aqueous phase extracted again with dichloromethane (200 ml), if necessary re-adjusting the pH to between 10.8 and 11.2 using 5% sodium hydroxide. The combined dichloromethane phases are washed with water (238 ml), if necessary re-adjusting the pH to between 10.8 and 11.2 using 5% sodium hydroxide. The phases are separated and the dichloromethane phase is evaporated to an oil under reduced pressure at less than 30° C. The oil is dissolved in toluene (176 ml) and the toluene removed by distillation under reduced pressure to a residual volume of 48-50 ml at less than 65° C. The residue is agitated at 60° C. and treated with n-heptane (210 ml), which has been pre-heated to 60° C. The resulting slurry of crystals is cooled to 0-5° C., and held within this temperature range for 1 hour. The (−) trans 4-(4′-fluorophenyl)-3-hydroxymethyl- 1-methylpiperidine is isolated by filtration, washed with chilled n-heptane (35 ml) and dried 35° C. under vacuum.

A yield of 20.6 g is obtained

### EXAMPLE 3

Repetition of Example 2 but increasing the volume of toluene employed in part i) to 100 ml results in a yield of 21.2 g

### EXAMPLE 4

Repetition of Example 2 but increasing the volume of toluene employed in part i) to 150 ml results in a yield of 20.8 g

### EXAMPLE 5

### Preparation of (−)ditoluoyltartaric Acid Salt of (−)Trans 4-(4′-fluorophenyl)-3-hydroxymethyl-l-methylpiperidine in 1:1 Toluene/Acetone

A solution of racemic trans 4-(4′-fluorophenyl)-3-hydroxymethyl-1-methylpiperidine (1.50 g) in toluene (10 ml) was warmed and stirred, and a solution of (−) di-toluoyltartaric acid (3.35g) in acetone (10 ml) was added. The mixture was allowed to cool gradually, and crystals formed within 5 minutes. The suspension was left to stand at ambient temperature overnight, then the white crystals were filtered, washed with acetone and dried under vacuum.

Yield 2.05 g.

1. A process for the manufacture of the (−) trans carbinol of formula (1) 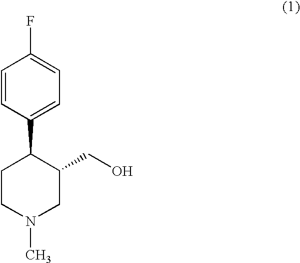
by a process comprising contacting a racemic mixture of the piperidine carbinol of structure (1) in solution with (−)-ditoluoyltartaric acid, crystallising the (−)-ditoluoyltartaric acid salt of the piperidine carbinol, isolating the crystalline salt and neutralising the crystalline salt to regenerate the (−) trans isomer of the piperidine carbinol and the (−)-ditoluoyltartaric acid,
which is characterised by one or more of the following steps:
(1) combining acetone solutions of the racemic piperidine carbinol and (−)-ditoluoyltartaric acid, typically in aqueous acetone and anhydrous acetone respectively, so that the combined solution contains 2-3 %wt/wt of water,
(2) combining a toluene solution of the racemic piperidine carbinol, or optionally a solution thereof in toluene and an auxiliary solvent, typically acetone, and a solution of (−)-ditoluoyltartaric acid in a compatible solvent, typically acetone,
(3) initiating the chiral salt crystallisation at from 30 to 40° C.,
(4) cooling the crystallisation mixture to from 3 to 7° C. before isolating the chiral salt,
(5) regenerating and extracting the (−) trans piperidine carbinol at a pH of from 10.5 to 11.5,
(6) forming a concentrated solution of the (−) trans piperidine carbinol in toluene, contacting the solution with heptane at 60-65° C., and cooling stepwise to crystallise the (−) trans piperidine carbinol.

2. A process according to claim 1 in which step (1) includes
a)i) dissolving trans 4-(4′-fluorophenyl)-3-hydroxymethyl-1-methylpiperidine in acetone with added water, which preferably contains between 4 and 6 %wt/wt of water, preferably at 30-35° C., and
ii) separately dissolving preferably 1.6 to 1.7 molar equivalents of (−)ditoluoyltartaric acid in preferably substantially the same volume of acetone as used in step a)i), preferably at 30-35° C.,

3. A process according to claim 1 or 2 in which step (1) includes bringing together the trans 4-(4′-fluorophenyl)-3-hydroxymethyl-1-methylpiperidine and (−) ditoluoyltartaric acid solutions as quickly as possible, preferably in less than 30 minutes, with rapid stirring, suitably at 120 rpm, preferably at a temperature between 30 and 40° C., more preferably between 35 and 40° C.

4. A process according to claim 1 in which step (2) includes addition of acetone and optionally water to the toluene solution of the racemic piperidine carbinol, before combining the toluene solution with a solution of (−)-ditoluoyltartaric acid in acetone.

5. A process according to any one of claims 1 to 4 in which step (3) includes holding the crystallisation mixture at between 35 and 40° C. for preferably 30 minutes to initiate crystallisation.

6. A process according to any one of claims 1 to 5 in which step (4) includes cooling the crystallisation mixture to between 3 and 7° C., for preferably 0.5 to 1.5 hours, more preferably for 1 hour, isolating the ditoluoyltartaric acid salt at between 3 and 7° C., washing with acetone, and optionally drying the ditoluoyltartaric acid salt,

7. A process according to any one of claims 1 to 6 in which step (5) includes dissolving the ditoluoyltartaric acid salt in water by the addition of an inorganic base, suitably aqueous sodium hydroxide, and extraction of the (−) trans carbinol into an organic solvent, suitably dichloromethane or toluene, preferably at a temperature in the range 15 to 25° C., at a pH in the range 10.5 to 11.5, more preferably in the range 10.8 to 11.2,

8. A process according to claim 7 in which includes concentrating a non-toluene solvent extract by evaporation of solvent under reduced pressure, preferably at less than 50° C., and replacing the solvent with toluene.

9. A process according to claim 7 or 8 which includes concentrating the toluene solution, preferably at less than 65° C., and mixing with heptane, preferably at 60-64° C.

10. A process according to claim 9 which includes cooling slowly, preferably to 40° C. over 1 hour to initiate crystallisation, optionally seeding, further cooling slowly, preferably to 20° C. over 1 hour, and finally cooling, preferably to 0° C.

11. A process according to any one of claims 1 to 10 which includes collecting the crystalline (−) trans 4-(4′-fluorophenyl)-3-hydroxymethyl-1-methylpiperidine preferably at 0° C., washing with heptane, and drying at preferably less than 35° C. until the loss in drying is preferably less than 0.5%.

12. A process for the large scale manufacture of the (−) trans carbinol of formula (1) by a resolution process with a chiral acid which comprises the following steps
a) forming respective solutions in acetone of trans 4-(4′-fluorophenyl)-3-hydroxymethyl-1-methylpiperidine and (−)ditoluoyltartaric acid, or of trans 4-(4′-fluorophenyl)-3-hydroxymethyl-1-methylpiperidine in toluene and (−)ditoluoyltartaric acid in acetone,
b) bringing together the trans 4-(4′-fluorophenyl)-3-hydroxymethyl-1-methylpiperidine and (−)ditoluoyltartaric acid solutions with stirring,
c) holding the mixture to initiate crystallisation,
d) cooling the crystallisation mixture, isolating the ditoluoyltartaric acid salt and washing with acetone, and optionally drying the ditoluoyltartaric acid salt,
e) dissolving the ditoluoyltartaric acid salt in water by the addition of an inorganic base, and extracting the (−) trans carbinol into an organic solvent,
f) concentrating the solvent extract by evaporation of solvent under reduced pressure, and replacing the solvent with toluene,
g) concentrating the toluene solution, and mixing with heptane,
h) cooling slowly to initiate crystallisation,
i) collecting the crystalline (−) trans 4-(4′-fluorophenyl)-3-hydroxymethyl-1-methylpiperidine, washing with heptane, and drying.

13. A process according to claim 12 characterised by one or more of the following steps:
a)i) dissolving trans 4(4′-fluorophenyl)-3-hydroxymethyl-1-methylpiperidine in acetone with added water, which preferably contains between 4 and 6 %wt/wt of water, preferably at 30-35° C., and
ii) separately dissolving preferably 1.6 to 1.7 molar equivalents of (−)ditoluoyltartaric acid in preferably substantially the same volume of acetone as used in step a)i), preferably at 30-35° C.,
b) bringing together the trans 4-(4′-fluorophenyl)-3-hydroxymethyl-1-methylpiperidine and (−)ditoluoyltartaric acid solutions with rapid stirring, suitably at 120 rpm, preferably at a temperature between 30 and 40° C., more preferably between 35 and 40° C.,
c) holding the crystallisation mixture at a temperature between 30 and 40° C. preferably between 35 and 40° C. for preferably 30 minutes to initiate crystallisation,
d) cooling the crystallisation mixture preferably to between 3 and 7° C. , for preferably 0.5 to 1.5 hours, more preferably for I hour, isolating the ditoluoyltartaric acid salt preferably at between 3 and 7° C., washing with acetone, and optionally drying the ditoluoyltartaric acid salt,
e) dissolving the ditoluoyltartaric acid salt in water by the addition of an inorganic base, suitably aqueous sodium hydroxide, and extraction of the (−) trans carbinol into an organic solvent, suitably dichloromethane or toluene, preferably at a temperature in the range 15 to 25° C., at a pH preferably in the range 10.5 to 11.5, more preferably in the range 10.8 to 11.2,
f) concentrating the solvent extract by evaporation of solvent under reduced pressure, preferably at less than 50° C., and replacing the solvent with toluene,
g) concentrating the toluene solution, preferably at less than 65° C., and mixing with heptane, preferably at 60-64° C.,
h) cooling slowly, preferably to 40° C. over 1 hour to initiate crystallisation, optionally seeding, further cooling slowly, preferably to 20° C. over 1 hour, and finally cooling, preferably to 0° C.,
i) collecting the crystalline (−) trans 4-(4′fluorophenyl)-3- hydroxymethyl-1- methylpiperidine preferably at 0° C., washing with heptane, and drying at preferably less than 35° C. until the loss in drying is preferably less than 0.5%.

14. A process according to any preceding claim in which the toluene solution of racemic trans 4-(4′-fluorophenyl)-3-hydroxymethyl-1-methylpiperidine is transferred from a previous stage in the production of paroxetine.

15. A process according to any one of claims 1 to 14 in which the (−) trans piperidine carbinol product is coupled with sesamol, then deprotected, to give paroxetine (2), and optionally forming a pharmaceutically acceptable salt of paroxetine.

16. A method of treating the Disorders which comprises administering an effective or prophylactic amount of paroxetine or paroxetine salt obtained using the process of claim 15 to a person suffering from one or more of the Disorders.
